(19) United States
(12) Patent Application Publication
(10) Pub. No.: US 2026/0158202 A1
(43) Pub. Date: Jun. 11, 2026

(54) ARTIFICIAL LUNG SYSTEM FOR DIRECT AIR CARBON CAPTURE

(71) Applicant: KING FAHD UNIVERSITY OF PETROLEUM AND MINERALS, Dhahran (SA)

(72) Inventors: Moataz Mohamed Hamed Abdelmoneim DOWAIDAR, Dhahran (SA); Ahmed Saleh Ahmad DALAQ, Dhahran (SA); Saravanan Sankaran SANKARAN, Dhahran (SA)

(73) Assignee: KING FAHD UNIVERSITY OF PETROLEUM AND MINERALS, Dhahran (SA)

(21) Appl. No.: 18/974,598

(22) Filed: Dec. 9, 2024

(51) Int. Cl. A61M 1/36; B01D 53/62; B01D 53/84

(52) U.S. Cl. CPC A61M 1/3666; B01D 53/62; B01D 53/84; B01D 2251/95; B01D 2257/504; B01D 2258/06

(57) ABSTRACT

An artificial lung system for direct air carbon capture. The artificial lung system includes a microfluidic device having a first connection point, a second connection point, and a third connection point, where the microfluidic device is configured to perform the direct air carbon capture. One or more air sacs each having an inlet and an outlet is connected to the microfluidic device. A multiple air channels are connected to the one or more air sacs and each air channel includes a valve. The valve of each air channel of the multiple air channels is configured to regulate microfluidic flows in the artificial lung system. An artificial heart is fluidly connected to the third connection point of the microfluidic device, where the artificial heart is configured to exchange a fluid in the microfluidic device.

### BACKGROUND

### Technical Field

The present disclosure is directed towards pulmonary assistive devices especially for use in treating respiratory illnesses, and more particularly, directed towards an artificial lung system for direct air carbon capture.

### Description of Related Art

The “background” description provided herein is to present the context of the disclosure generally. Work of the presently named inventors, to the extent it is described in this background section, as well as aspects of the description that may not otherwise qualify as prior art at the time of filing, are neither expressly nor impliedly admitted as prior art against the present invention.

With the constant rise in population, pulmonary and respiratory illnesses are increasing. As per recent numbers, approximately 454 million human beings suffer from chronic respiratory disease (CRD), a number of them resulting in respiratory failure. Hence, efficient and patient friendly respiratory support devices are required to mitigate aforementioned health crisis. One such device is the mechanical ventilator, improved by numerous methods, calculations, and changes in order to enhance the effectiveness of the mechanical ventilator. However, ventilator associated events (VAEs) are unavoidable when using a mechanical ventilator. VAEs include, but may not be limited to, ventilator-associated pneumonia (VAP), sepsis, septic shock, acute respiratory distress syndrome (ARSD), atelectasis, and pulmonary edema. Further, lung transplantation is another method of supporting patients suffering from respiratory failure. However, the number of applicants for lung transplants increased by 11 % between 1997 and 2007. The demand exceeds the supply since there is an enormous disparity between the number of people in need of lung transplants and the quantity of available lungs from donors. Furthermore, only 18% of the donated lungs were transplanted in 2006, and about 81% were not retrieved. This mismatch was attributed to poor organ function, which caused an even larger gap between the number of lungs that were required and those that were available. Therefore, researchers have considered artificial lungs as a means of transitioning to transplantation in addition to a replacement organ. An effective artificial lung may be utilized supportively after a transplant or to upgrade mechanically supported breathing.

Traditionally, artificial lung technologies have only been used with cardiopulmonary bypass and extracorporeal membrane oxygenation (ECMO). However, ECMO has innate difficulties and associated drawbacks. ECMO takes a long time, requires considerable staff and supervision, and the ECMO apparatus is made up of tubes and cannulas that may burst and have catastrophic consequences. Multiple transfusions are necessary for ECMO treatment, which is non-ambulatory and has additional side effects, such as, contamination, erythrocyte and platelet obliteration, and embolization of cylinder sections. Hence, recent developments have moved away from ECMO treatment for the reasons mentioned above. Further, an intravascular oxygenator (IVOX) is yet another respiratory support device. In an IVOX, gas enters and leaves the framework through a small skin entry point, a vacuum siphon draws oxygen through the film oxygenator's filaments when it is embedded into the vena cava. Another implementation involves blood blending across the gas exchange component using a vacuum siphon. However, IVOX is insufficient with respect to surface region, gas exchange capacity, and cannot fulfill the needs of a patient undergoing or recuperating from a lung transplant. However, all the existing technologies have certain drawbacks. Therefore, artificial lungs are considered to be more functional and efficient substitute, since fewer gadgets are attached to the pulmonic flow that may support lung digestion, empower proper gas exchange, and be completely perfused by the right ventricle. In addition, artificial lungs may support function in ambulatory patients, permitting almost unrestricted patient mobility and activity, which is crucial.

Accordingly, it is one object of the present disclosure to provide an artificial lung system for direct air carbon capture, that may circumvent the aforementioned drawbacks of the known systems.

### SUMMARY

In an exemplary embodiment, an artificial lung system for direct air carbon capture is described. The artificial lung system includes a microfluidic device having a first connection point, a second connection point, and a third connection point, where the microfluidic device is configured to perform the direct air carbon capture. The artificial lung system further includes one or more air sacs including a posterior sac and an anterior sac each having an inlet and an outlet, a plurality of air channels each including a valve, wherein the valve of each air channel of the plurality of air channels is configured to regulate microfluidic flows in the artificial lung system; and an artificial heart fluidly connected to the third connection point of the microfluidic device and configured to exchange a fluid in the microfluidic device, wherein the inlet of the posterior sac is connected to a first air channel of the plurality of air channels via a first valve and the outlet of the posterior sac is connected to the first connection point of the microfluidic device; and wherein the inlet of the anterior sac is connected to the second connection point of the microfluidic device and the outlet of the anterior sac is connected to a second air channel of the plurality of air channels via a second valve. In some embodiments, each air sac of the plurality of air sacs further includes a plurality of biomimetic soft tissues and a plurality of biomimetic hard tissues.

In some embodiments, each biomimetic soft tissue of the plurality of biomimetic soft tissues includes a spider silk.

In some embodiments, each biomimetic hard tissue of the plurality of biomimetic hard tissues includes at least one carbonic anhydrase immobilized in a solid matrix.

In some embodiments, the carbonic anhydrase is a cadmium-containing carbonic anhydrase (CDCA).

In some embodiments, the plurality of air sacs includes a posterior sac and an anterior sac.

In some embodiments, the inlet of the posterior sac is connected to a first air channel of the plurality of air channels via a first valve and the outlet of the posterior sac is connected to the first connection point of the microfluidic device; and where the inlet of the anterior sac is connected to the second connection point of the microfluidic device and the outlet of the anterior sac is connected to a second air channel of the plurality of air channels via a second valve.

In some embodiments, the first valve and the second valve are air solenoid valves.

In some embodiments, the artificial lung system is configured such that the first valve is open, and the second valve is closed during an inhale phase.

In some embodiments, the artificial lung system is configured such that the first valve is closed, and the second valve is open during an exhale phase.

In some embodiments, the one or more air sacs is an origamilike bellow, where the origamilike bellow includes the plurality of biomimetic soft and hard tissues.

In some embodiments, the fluid includes one or more genetically engineered microorganisms.

In some embodiments, the artificial lung system further includes a peristaltic pump connected to the plurality of air channels, where the peristaltic pump is configured to control an air flow of the artificial lung system.

In some embodiments, the microfluidic device is a flexible microfluidic device having a shape of alveoli.

In some embodiments, the microfluidic device includes a plurality of distribution channels each having a first end and a second end, a first distributor connected to the first end of each distribution channel of the plurality of distribution channels, where the first distributor includes a plurality of first connectors connected to the first connection point of the microfluidic device, and a second distributor connected to the second end of each distribution channel of the plurality of distribution channels, where the second distributor includes a plurality of second connectors connected to the second connection point of the microfluidic device, where the plurality of distribution channels is configured for gas exchange.

In some embodiments, each distribution channel of the plurality of distribution channels is semi-elliptical and where the plurality of distribution channels is arranged in an approximately concentric circular shape.

In some embodiments, the one or more air sacs is configured to support a unidirectional airflow.

The foregoing general description of the illustrative present disclosure and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is a schematic diagram of an artificial lung system illustrating an inhalation phase, according to certain embodiments;

FIG. 1B is a schematic diagram of the artificial lung system illustrating an exhalation phase, according to certain embodiments;

FIG. 2A illustrates a schematic diagram of an artificial lung system including an origamilike bellow, according to certain embodiments;

FIG. 2B is a schematic illustration of a molecular structure of a cadmium containing carbonic anhydrase, according to certain embodiments;

FIG. 3A illustrates a schematic diagram of an artificial lung system including a peristaltic pump, according to certain embodiments;

FIG. 3B illustrates a schematic diagram of a flexible microfluidic device, according to certain embodiments;

FIG. 4A illustrates a schematic diagram depicting a parallel configuration of the artificial lung system of FIG. 1A, according to certain embodiments;

FIG. 4B illustrates a schematic diagram depicting a series configuration of the artificial lung system of FIG. 1A, according to certain embodiments;

FIG. 5A is an optical image of an enrichment culture media FB1, according to certain embodiments;

FIG. 5B is an optical image of an enrichment culture media FB2, according to certain embodiments;

FIG. 5C is an optical image of an enrichment culture media FB3, according to certain embodiments;

FIG. 6A is an optical image of an enrichment culture media with plant oil, according to certain embodiments;

FIG. 6B is another optical image of an enrichment culture media with plant oil, according to certain embodiments;

FIG. 7A is an optical image of a sample plated onto agar formation brine medium with crude oil (1% v/v) as carbon source, according to certain embodiments;

FIG. 7B is an optical image of the samples after an incubation period of 3 days at 30° C., according to certain embodiments;

FIG. 7C is another optical image of the samples after an incubation period of 3 days at 30° C., according to certain embodiments;

FIG. 8A is an optical image of FB1 sample after turning turbid depicting a dark layer of crude oil indicating microbial growth of substrate, according to certain embodiments;

FIG. 8B is an optical image of FB2 sample after turning turbid depicting a dark layer of crude oil indicating microbial growth of substrate, according to certain embodiments;

FIG. 8C is an optical image of FB3 sample after turning turbid depicting a dark layer of crude oil indicating microbial growth of substrate, according to certain embodiments;

FIG. 8D is an optical image of two sample after turning turbid depicting a dark layer of crude oil indicating microbial growth of substrate, according to certain embodiments;

FIG. 9A is an optical image of a borosilicate flat bottom flask having a volume of 5000 ml and containing the culture media enriched with carbon dioxide (CO₂), according to certain embodiments;

FIG. 9B is an optical image of the borosilicate flat bottom flask with the culture media undergoing deoxygenation by CO₂, according to certain embodiments;

FIG. 9C is an optical image of the borosilicate flat bottom flask with the culture media maintained at least 90 days, according to certain embodiments;

FIG. 10A is an optical image of an Erlenmeyer flask with a volume of 2500 ml, including the culture media, according to certain embodiments; and

FIG. 10B is an optical image of the Erlenmeyer flask and its contents undergoing deoxygenation by nitrogen gas (N₂), according to certain embodiments.

### DETAILED DESCRIPTION

In the drawings, reference numerals designate identical or corresponding parts throughout the several views. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of the present disclosure are directed towards an artificial lung system configured for direct air carbon capture. The system as described herein is designed to substitute a mechanical ventilator in healthcare applications and support patients and individuals with respiratory or pulmonary failure. In accordance with the present disclosure, the artificial lung system is configured to be a biomimetic system. Further, the artificial lung system may be inspired by high efficiency respiratory system of a hummingbird. As such, by combining aspects such as an architecture of a respiratory system of a hummingbird with the capabilities of spider silk, a carbonic anhydrase, a microfluidic system, and genetically engineered microorganisms performing the Calvin-Benson pathway, the present disclosure provides an efficient and sustainable solution for direct air carbon capture at large scale deployments and biomedical respiratory support at individual level employment. In certain implementations, the microfluids as described in the present disclosure may be utilized for enhanced oil recovery from an oil reservoir. This may be achieved via engineered microorganisms, present in the microfluids.

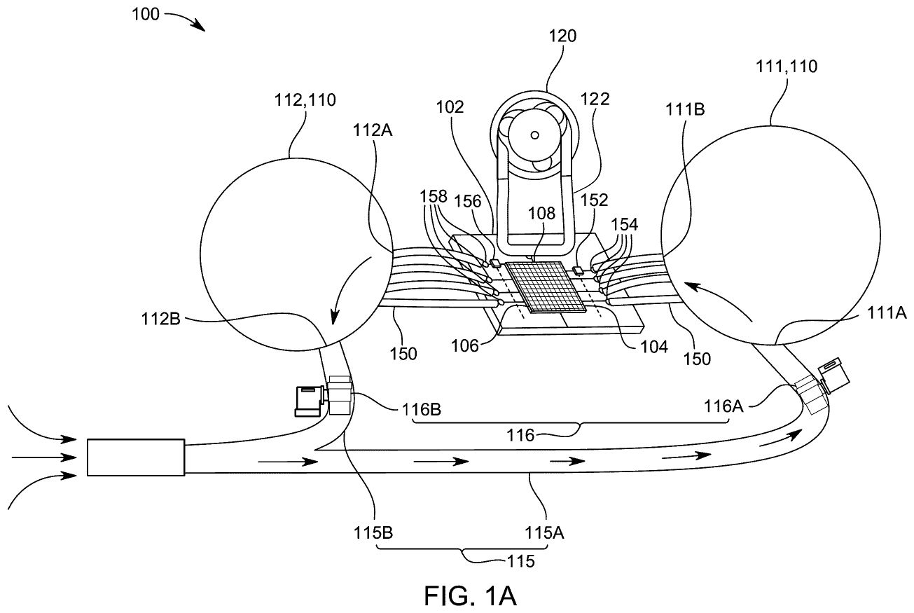

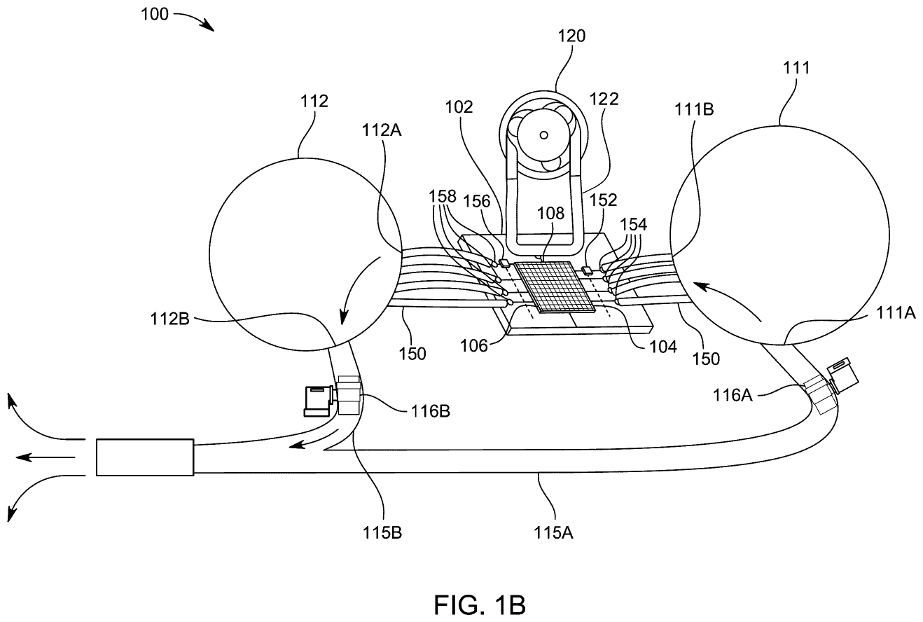

Referring to FIG. 1A, illustrated is a schematic diagram of an artificial lung system 100 depicting an inhalation phase, according to certain embodiments. In particular, FIG. 1A illustrates a plurality of components included in the artificial lung system 100. The artificial lung system 100 may be interchangeably referred to as “the system 100”, throughout the present disclosure. In some embodiments, the system 100 includes a microfluidic device 102 having a first connection point 104, a second connection point 106, and a third connection point 108. The first, second and third connection points 104, 106, and 108 (e.g., fluid inlets and/or outlets) are configured to establish a fluid communication between a plurality of components included in the system 100 and the microfluidic device 102. In one embodiment, the microfluidic device 102 may be a cuboidal structure which defines a volume for storage and processing of a microfluid. The first connection point 104 and the second connection point 106 are preferably disposed parallelly opposite to each other, as such, the first and the second connection points 104, 106 are placed in-front of each other at a predetermined distance. The predetermined distance may be dependent on a size of the microfluidic device 102. Further, the third connection point 108 is disposed perpendicular to the first and the second connection points 104, 106. In another embodiment, the microfluidic device 102 may be of any other suitable shape such as a cube, a sphere, a concentric circular shape, and the like. In some embodiments, the microfluidic device 102 may be a flat and square (e.g. a puck-like shape) device comprising a bifurcated network of microfluidic channels. Preferably the channels are oriented such that fluid flow occurs in a single direction from one side of the microfluidic device to the other. As shown in FIG. 1A the fluid inlets and outlets are preferably positioned on flat edge faces of the microfluidic device with each inlet directly opposite a corresponding outlet such that each inlet/outlet pair represents a fluid flow path that is parallel to the fluid flow paths of the other inlet/outlet pairs. Channels within the microfluidic device preferably have flat tops and flat bottoms, and tapered or convex sides. The microfluidic channels in the bifurcated network may have a variable diameter or a fixed diameter to adjust the flow rate of the device. In another embodiments, the microfluidic device may be similar to the exchange device disclosed in US 2014/0193799 A1 which is incorporated herein by reference in its entirety. In yet other embodiments, the microfluidic channels may comprise channel walls defined by flow disturbance elements such as micro-grooves, micro-protrusion obstacles, and similar. The flow disturbance elements may be rectangular, triangular, and/or circular structure of variable or fixed size. Examples of the microfluidic device 102 according to some embodiments may be found in Cheng et al., Microfluidic-based chemical absorption technology for CO2 capture: Mass transfer dynamics, operating factors and performance intensification, Renewable and Sustainable Energy Reviews, Volume 181, 113357 (2023), which is incorporated herein by reference in its entirety. The microfluidic device 102 is configured to perform direct air carbon capture (DAC). In general, DAC is the use of chemical or physical processes to extract carbon dioxide (CO₂) directly from atmospheric or ambient air. In case the extracted CO₂ is sequestered in safe long-term storage, the overall process will achieve carbon dioxide removal from the atmosphere and thus function in a negative emissions technology (NET) capacity.

The system 100 further includes one or more air sacs 110. In some embodiments, the system 100 may include a posterior sac 111 and an anterior sac 112. Further, each of the posterior sac 111 and the anterior sac 112 has an inlet and an outlet, as such, the posterior sac 111 has an inlet 111A, and an outlet 111B and the anterior sac 112 has an inlet 112A and an outlet 112B. In some embodiments, the one or more air sacs 110 are connected to the microfluidic device 102. In other words, the one or more air sacs 110 are fluidly coupled with the microfluidic device 102 in order to exchange air. In some embodiments, the one or more air sacs 110 are biomimetic in design. Design of the one or more air sacs 110 is inspired by natural biological structures. In general, biomimetics or biomimicry is an emulation of models, systems, and elements of nature for the purpose of solving complex human problems. As such, each air sac of the one or more air sacs 110 further includes a plurality of biomimetic soft tissues and a plurality of biomimetic hard tissues. In some embodiments, the one or more air sacs 110 is configured to support a unidirectional airflow. In another embodiments, the volume of each air sac of the one or more air sacs 110 can be selected to give a prosthetic lung that substitutes effectively for a natural lung, for example, between 0.5 and 2 liters of air on each breath. In yet another embodiments, the one or more air sacs 110 comprises wholly or partly of an elastic material such as a plastic polymer or rubber material. Suitable elastic air sac materials include silicone rubbers. In general, unidirectional airflow is controlled airflow through an entire cross-section of a conduit with a steady velocity and approximately parallel streamlines. Hence, the one or more air sacs 110 supports the unidirectional airflow as it may result in a direct transport of gas from one end to other. Further, unidirectional airflow may also maintain almost cleanroom conditions inside the system 100, preventing the system 100 from contamination. In some embodiments, the one or more air sac 110 mimic air sacs of an avian respiratory system, especially a hummingbird's. Unlike mammals, birds possess air sacs. These sacs, especially prominent in hummingbirds, enable unidirectional airflow, ensuring constant oxygenation during both inhalation and exhalation. The air sacs in the bird's body stretch into the bones and may be found all around the abdominal cavity. This is one of the positive aspects of the bird's respiratory system, which allows gas exchange to occur throughout the whole body. However, unlike human lungs, avian lungs do not expand and contract in response to changes in air pressure.

Inhalation and exhalation each make up half of a bird's respiratory cycle, and the two cycles are separated by a pause. During inhalation, air travels via the nares into the nasal cavity before continuing on to the trachea through the larynx. Afterward, it travels via the syrinx and then partitions into the back air sacs and the other air sacs until eventually reaching the lungs.

The lungs are then responsible for gas exchange by diffusion, with oxygen diffusing into the blood of the bird and carbon dioxide diffusing from the blood into the lungs.

During exhalation, air goes from the back air sacs into the lungs, where the process of gas exchange occurs. Then, when the next round of inhalation takes place, the gas is transferred from the lungs into the cranial air sacs. During the second pattern of exhalation, the air is removed from the body through the nostrils after traveling from the cranial air sacs to the trachea.

Furthermore, the system 100 includes a plurality of air channels 115. In particular, the plurality of air channels 115 include a first air channel 115A and a second air channel 115B. In general, an air channel refers to a tubular conduit, designed to carry air or a fluid throughout a length of the air channel. The first and the second air channels 115A, 115B are configured to be flexible in construction. The plurality of air channels 115 is configured to be connected to the one or more air sacs 110. As such, the first air channel 115A is configured to fluidly couple with the posterior sac 111 and the second air channel 115B is configured to fluidly couple with the anterior sac 112. Further, in order to regulate fluid flow, each air channel of the plurality of air channels 115 includes a valve 116. In some embodiments, the first air channel 115A includes a first valve 116A and the second air channel 115B includes a second valve 116B. The first and second valves 116A, 116B are configured to be positioned transversely along a cross-section of each of the plurality of air channels 115. The valve 116 of each air channel 115A, 115B of the plurality of air channels 115 is configured to regulate microfluidic flows in the artificial lung system 100.

In some embodiments, the inlet 111A of the posterior sac 111 is connected to the first air channel 115A of the plurality of air channels 115, via the first valve 116A. The connection between the first air channel 115A and the first valve 116A is configured to be air-tight and water-tight, in order to keep fluids from leaking out of the system 100. Further, the outlet 111B of the posterior sac 111 is connected to the first connection point 104 of the microfluidic device 102. In other words, the microfluidic device 102 is configured to be fluidly coupled with the posterior sac 111 via the first connection point 104. However, in some implementations of the system 100, the microfluidic device 102 may be coupled with the posterior sac 111 via different connection points depending on a requirement of a user of the system 100. Furthermore, the anterior sac 112 is fluidly coupled with the microfluidic device 102 in a similar manner as described above. In other words, the inlet 112A of the anterior sac 112 is connected to the second connection point 106 of the microfluidic device 102 and the outlet 112B of the anterior sac 112 is connected to the second air channel 115B of the plurality of air channels 115, via the second valve 116B. As such, fluid connections and joints formed between the aforementioned components are air-tight and water-tight. In some embodiments, the first valve 116A disposed between the first air channel 115A and the posterior sac 111, and the second valve 116B disposed between the anterior sac 112 and the second air channel 115B, are air solenoid valves, otherwise referred to as “pneumatic solenoid valves”. In general, pneumatic solenoid valves are electromechanical devices that control a flow of air. Pneumatic solenoid valves are used for controlling pneumatic actuators such as cylinders, pneumatic motors, diaphragms, and tubes. Pneumatic solenoid valves form auxiliary air circuits, using actuators, to control equipment where they are disposed. As described with respect to FIG. 1A, the artificial lung system 100 is in the inhalation phase. The artificial lung system 100 is configured such that the first valve 116A is open, and the second valve 116B is closed, during the inhale phase.

Further, in some embodiments, the artificial lung system 100 includes an artificial heart 120. In particular, the artificial heart 120 is connected to the third connection point 108 of the microfluidic device 102. The artificial heart 120 is positioned between the posterior sac 111 and the anterior sac 112, as such, the artificial heart 120 is fluidly coupled with the third connection point 108 of the microfluidic device 102. In some embodiments, the artificial heart 120 is configured to exchange a fluid in the microfluidic device 102. In some embodiments, the artificial heart 120 is connected to the third connection point 108 via a gas-permeable membrane tube 122 serves to provide gas exchange therebetween. The gas-permeable membrane 122 may be a gas-membrane polymer membrane or any other material that is sufficiently robust to withstand forces applied to the gas-permeable membrane 122 and to contain the fluid passing through the gas-permeable membrane 122 while providing transfer of gas through the gas-permeable membrane 122. In some embodiments, the gas-permeable membrane 122 may be in direct contact with the microfluidic device 102 at the third connection point 108 in the manner that the transfer of gas through the gas-permeable membrane is maximized. In an embodiment of the present disclosure, the fluid includes one or more genetically engineered microorganisms. Further details pertaining to the fluid are explained in detail with experimental evidence in the “examples” section of the present disclosure. In some embodiments, the artificial heart 120 may be a blood pump generally used in extracorporeal membrane oxygenation (ECMO). In another embodiments, the artificial heart 120 may be a roller pump or centrifugal pump. A roller pump displaces blood through flexible tubing located inside a curved raceway to generate forward flow proportional to the pump speed and tubing size. Centrifugal pumps generate a pressure differential across the pump head via spinning pump components and centrifugal force, resulting in negative pressure in the drainage tubing and subsequent blood flow.

The microfluidic device 102 further includes a plurality of distribution channels 150 each having a first end and a second end. Further, the microfluidic device 102 includes a first distributor 152 connected to the first end of each distribution channel of the plurality of distribution channels 150, as such, the first distributor 152 includes a plurality of first connectors 154 connected to the first connection point 104 of the microfluidic device 102. The microfluidic device 102 includes the first distributor 152 in order to equally distribute an amount of the fluid present in the volume defined by the microfluidic device 102. The first distributor 152 helps in maintaining a regular flow to the first end of the plurality of distribution channels 150. In a similar manner, the microfluidic device 102 includes a second distributor 156 connected to the second end of each distribution channel of the plurality of distribution channels 150, as such, the second distributor 156 includes a plurality of second connectors 158 connected to the second connection point 106 of the microfluidic device 102. In some embodiments, the plurality of distribution channels 150 is configured for gas exchange, in order to perform direct air carbon capture via the system 100.

Referring to FIG. 1B, illustrated is a schematic diagram of the artificial lung system 100 showing an exhalation phase, according to certain embodiments. As shown in FIG. 1B, the artificial lung system 100 is explained with respect to the exhalation phase after the completion of inhalation phase as described in FIG. 1A. The artificial lung system 100 of FIG. 1B is configured such that the first valve 116A is closed, and the second valve 116B is open during the exhalation phase. The artificial lung system 100, in its inhalation and exhalation phase, closely resembles biological breathing which occurs in humans and avians.

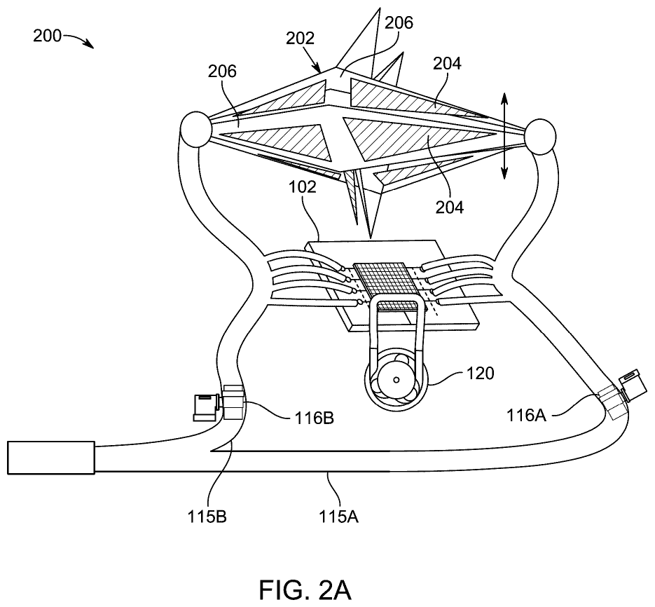

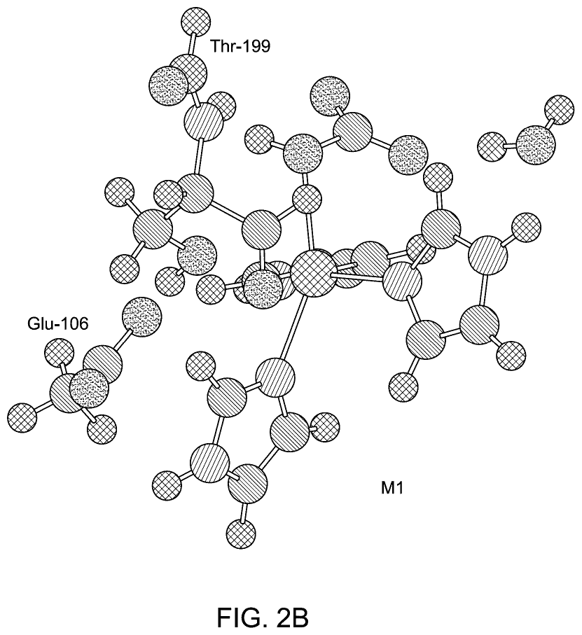

Referring to FIG. 2A, a schematic diagram of an artificial lung system 200 including an origamilike bellow 202 is illustrated, according to certain embodiments. In particular, FIG. 2A illustrates an exemplary design of the one or more air sacs 110 in the form of the origamilike bellow 202. For an illustration purpose, FIG. 2A depicts only one origamilike bellow 202 as the air sac, however, a person with ordinary skill in the art would anticipate that one or more origamilike bellows 202 may be included in the artificial lung system 200 in order to meet a requirement of a practical application where the artificial lung system 200 may be employed.

The origami like bellow 202 includes a plurality of soft and hard tissues, namely, the biomimetic soft tissue 204 and the biomimetic hard tissue 206. In some embodiments, the biomimetic soft and hard tissues may be 3D biomimetic porous collagen scaffold, photopolymerizable polydimethylsiloxane (PDMS) resin, or other synthetic biomaterials for tissue engineering (TE) such as polylactic acid, polyhydroxyalkanoates, hyaluronic acid, alginate, cellulose, chitosan, fibroin, antimicrobial peptides, and similar. In particular, the plurality of soft tissues 204 is configured to make up a movable (compressible) part of the origami like bellow 202 whereas the plurality of hard tissues 206 are utilized to provide additional structural rigidity to the origamilike bellow 202. In one embodiment, the origami like bellow 202 is designed to have a broader middle section and narrow ends. In other words, the origami like bellow 202 is dimensionally larger at a middle section and dimensionally narrower at the ends. Further, in one embodiment, each of the plurality of biomimetic soft tissues 204 includes a spider silk. In general, silk is a type of sinewy protein created in specialized epithelial cells in organs of various spiders. These cells have been observed in a plurality of species of spiders. Further, spider silk is earth stable, nonimmunogenic, bioresorbable, biocompatible, biodegradable, and morphologically adaptable. Spider silk may also immobilize developmental chemicals by adjusting amino corrosive arrangements. Spider silk has been used in a range of wound healing approaches. Local spider silk filaments can promote development of epidermal layers over strands and elevate keratinization for in-vitro skin models. The aforementioned qualities of spider silk are in addition to distinctive strength and resilience features already considered, regarding use of spider silk as biomimetic soft tissue in the artificial lung systems 100, 200. Furthermore, makeup of each spider silk protein is determined by precise amino corrosive arrangements and movements of arachnid from which the silk was initially extricated. Examination of standout material qualities of the arachnid silk has provided enablement for using spider silk in diverse biomedical applications. In the present disclosure, spider silk has been used in biomimetic soft tissues 204 of the artificial lung systems 100, 200 since regular replacement of spider silk wound dressings minimizes the development of unfavorable inflammatory responses. In another embodiment, the plurality of biomimetic soft tissues 204 may include another bioavailable fiber. Furthermore, each biomimetic hard tissue 206 of the plurality of biomimetic hard tissues includes at least one carbonic anhydrase immobilized in a solid matrix. In some embodiments, the solid matrix is spider silk.

In general, carbonic anhydrases or carbonate dehydratases form a family of enzymes that catalyze the interconversion between carbon dioxide and water, and the dissociated ions of carbonic acid. An active site of most carbonic anhydrases contains a zinc ion. Carbonic anhydrases are therefore classified as metalloenzymes. This metalloenzyme maintains acid-base balance and helps transport carbon dioxide. Moreover, referring to FIG. 2B, a schematic illustration of a molecular structure of the carbonic anhydrase is illustrated, according to certain embodiments. In particular, the carbonic anhydrase is a cadmium-containing anhydrase (CDCA). Further, CDCA trades cadmium for zinc when zinc becomes more available to a phytoplankton than cadmium. This is a significantly favorable characteristic of CDCA versus other carbonic anhydrases.

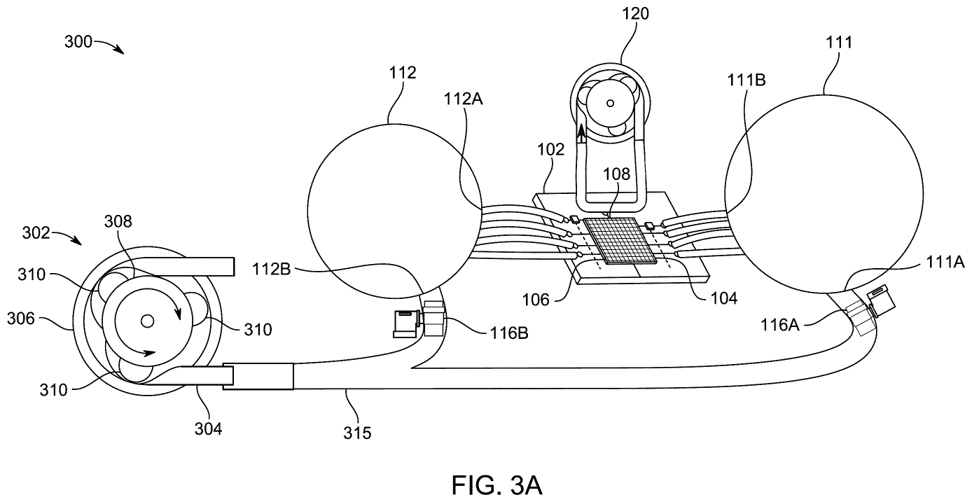

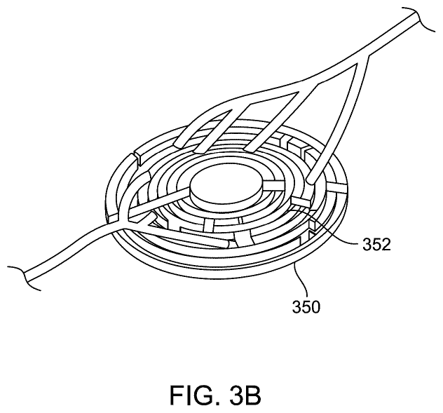

Referring to FIG. 3A, a schematic diagram of an artificial lung system 300 is illustrated, according to certain embodiments. The artificial lung system 300 closely resembles the artificial lung system 100 of FIG. 1A. Further, the key components of the artificial lung system 300 are identical to the key components of the artificial lung system 100 of FIG. 1A. Furthermore, the key components of the artificial lung system 300 interact in a similar manner as the key components of artificial lung system 100 of FIG. 1A. However, the artificial lung system 300 has certain advancements over the artificial lung system 100 of FIG. 1A, as detailed in the subsequent paragraph(s).

In some embodiments, the artificial lung system 300 includes a peristaltic pump 302. The peristaltic pump 302 is connected to a plurality of air channels 315 and the peristaltic pump 302 is configured to control an air flow of the artificial lung system 300. In general, the peristaltic pump is a type of positive displacement pump used for pumping a plurality of fluids. The fluid (air in the present disclosure) is contained in a flexible tube 304 fitted inside a circular pump casing 306. Further, the peristaltic pump 302 works through rotary motion. However, a linear peristaltic pump may be used in some implementations of the present disclosure. Furthermore, the peristaltic pump 302 includes a pump rotor 308 that further includes a plurality of rollers 310 attached to an external circumference of the pump rotor 308. The plurality of rollers 310 compresses the flexible tube 304 as they rotate via the pump rotor 308. As such, a part of the flexible tube 304 under compression is closed, forcing the air to move through the flexible tube 304. In addition, as the flexible tube 304 opens to its natural state after the plurality of rollers 310 pass, more air is drawn into the flexible tube 304. This process is generally known as peristalsis.

Referring to FIG. 3B, a schematic diagram of a flexible microfluidic device 350 is illustrated, according to an embodiment of the present disclosure. In some embodiments, the flexible microfluidic device 350 has a shape of alveoli. In general, alveoli are minuscule, balloon-shaped air sacs located at the end of bronchioles, the branch-like tubes in human or biological lungs. The alveoli move oxygen and carbon dioxide (CO₂) molecules into and out of bloodstream of organisms. In some implementations of the present disclosure, the flexible microfluidic device 350 may be used in the artificial lung systems 100, 200, 300 in place of the microfluidic device 102, shown in FIGS. 1A, 2A, and 3A, which is rigid. In some embodiments, the flexible microfluidic device 350 includes a plurality of distribution channels 352 which are arranged in an approximately concentric circular shape. The plurality of distribution channels 352 may be further connected with the flexible microfluidic device 350 using a plurality of connectors, as such the flexible microfluidic device 350 is fluidly coupled with the air sacs 110. In some embodiments, each distribution channel of the plurality of distribution channels 352 is semi-elliptical. In some embodiments, a cross section of the distribution channel 352 may be circular, semi-circular, or any other shape known in the art. In particular, the semi-elliptical shape provides the artificial lung system 300 with necessary flow rate required for efficient and effective carbon dioxide exchange with the atmosphere. The semi-elliptical shape is also associated with a laminar flow of fluids in medical support devices.

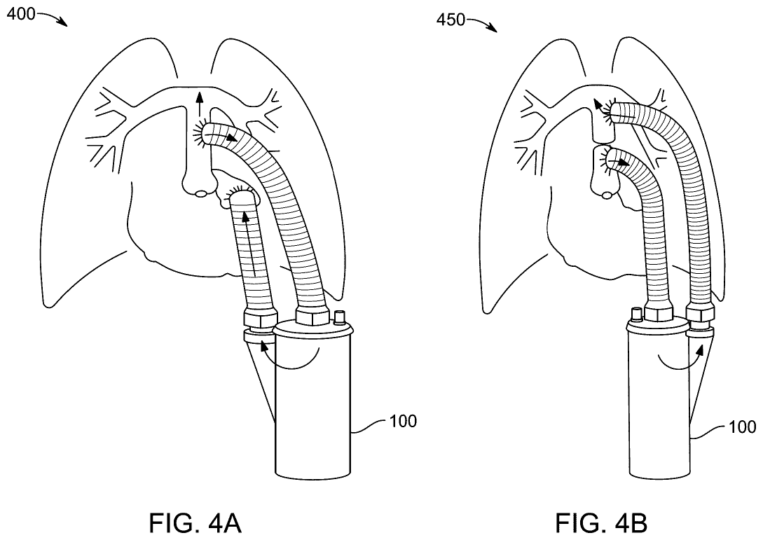

The artificial lung systems 100, 200, 300 may be employed in multiple configurations to support a patient during a pulmonary illness. Referring to FIG. 4A, a schematic diagram depicting a parallel configuration 400 of the artificial lung system 100 is illustrated, according to certain embodiments. Referring to FIG. 4B, a schematic diagram depicting a series configuration 450 of the artificial lung system 100 is illustrated, according to certain embodiments. The parallel configuration 400 may be useful in cases where biological lungs of the patient are required to be entirely bypassed. However, the series configuration 450 may be used in cases where the biological lungs and associated functions are necessary for survival of the patient. The parallel configuration 400 bypasses the biological lungs completely while the series configuration 450 is used in addition to the biological lungs, where the artificial lung system 100 in series configuration 450 draws blood from a right ventricle of the patient.

### EXAMPLES

The following examples demonstrate a method of synthesis of one or more genetically engineered microorganisms, for use in the artificial lung systems 100, 200, 300. The examples are provided solely for illustration and are not to be construed as limitations of the present disclosure, as many variations thereof are possible without departing from the spirit and scope of the present disclosure.

### Example 1: Enrichment Culture Media

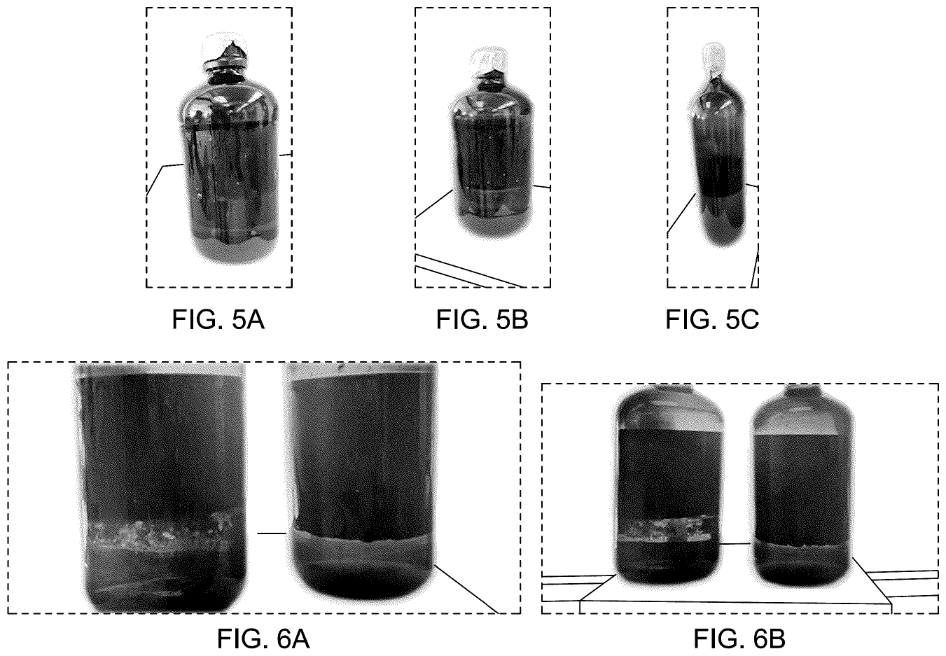

An enrichment culture medium was prepared by using Saudi synthetic brine solution, NaCl concentration of 2.574603 mole per liter (mol/L). The formation brine solution was prepared by adding NaCl, 150.46 grams (g); Na₂SO₄, 0.5175 g; NaHCO₃, 0.4874 g; CaCl₂ dihydrate, 69.823 g; and MgCl₂(6H₂O), 20.395 g to 758.32 ml distilled water. The medium was sterilized by autoclaving at 121° C. for 20 minutes (min). Further, after reaching the room temperature, crude oil was added to the above solution at 25% volume by volume (v/v) and labeled as FB1, as shown in FIG. 5A.

### Example 2: Medium with Plant Oil and Sucrose

A formation brine solution was prepared by adding NaCl, 150.46 g; Na₂SO₄, 0.5175g, NaHCO₃, 0.4874 g; CaCl₂ dihydrate, 69.823 g; MgCl₂(6H₂O), 20.395 g, and sucrose 100 g to 758.32 ml distilled water and 25% (v/v) commercial plant fatty oil products, with a total fat of 100 g, where, saturated fat was 15.35 g, monounsaturated fat was 20.45g, polyunsaturated fat was 30.55g, and trans-fat was 0.5 g per 100 mL, were purchased from the local markets. The medium was sterilized by autoclaving at 121° C. for 20 min. Further, after reaching room temperature, crude oil was added to the above solution at 25% volume by volume (v/v) and labeled as FB2, as shown in FIG. 5B.

### Example 3: Medium with Plant Oil

The formation brine solution was prepared by adding NaCl, 150.46 g; Na₂SO₄, 0.5175 g; NaHCO₃, 0.4874 g; CaCl₂ dihydrate, 69.823 g; MgCl₂(6H₂O), 20.395 g; to 758.32 ml distilled water and 25% v/v commercial plant fatty oil products purchased from the local markets. The medium was sterilized by autoclaving at 121° C. for 20 min. Further, after reaching room temperature, crude oil was added to the above solution at 25% volume by volume (v/v) and labeled as FB3, as shown in FIG. 5C

All the medium was assessed by using amber Boston round glass bottle (2.5 L) with a working volume of 2 L. Further, the medium was continuously stirred at 200 revolutions per minute (rpm) by using a magnetic stirrer at room temperature of about 20° C. The condition of the culture medium was maintained, and the flasks were sealed with adhesive tape for at least 90 days, as shown in FIGS. 6A-6B. In addition, 10 ml of samples (every ten days) were collected for analysis, which included isolating the individual strains, metagenomics and GC-MS analyses. Outstanding samples were stored at −20° C. until further tests were conducted.

### Example 4: Isolation of Individual Strains

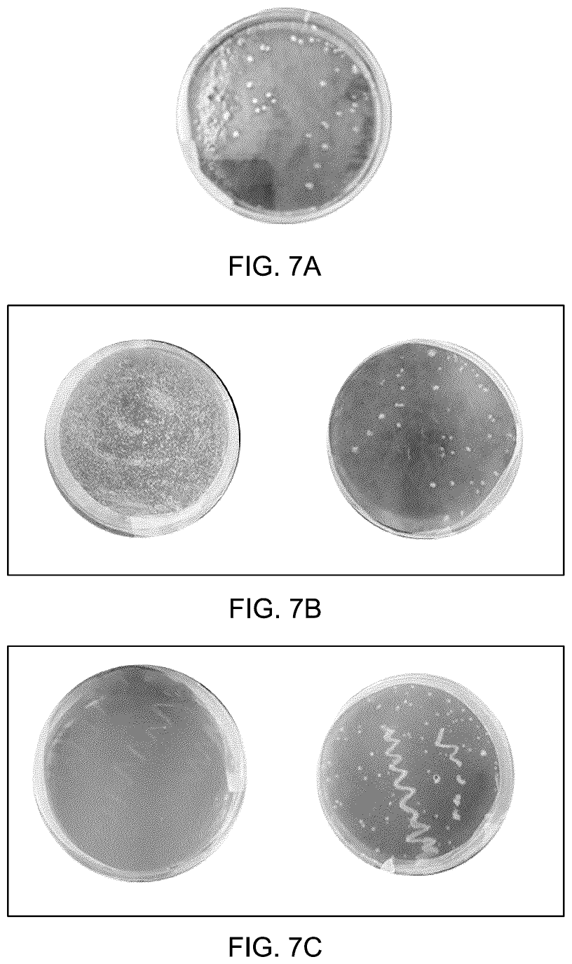

Enrichment culture sample of 1 ml was used for isolation on solid medium. Referring to FIGS. 7A-7C, aliquots of 0.1 ml (1 to 10) having 10 dilutions, were plated onto agar formation brine medium with crude oil 1% v/v as carbon source. The plates were incubated at 30° C. for 3 days, until colony formation. Single colonies were picked and used for screening. Individual colonies were purified by repeated streaking on agar formation brine medium containing 1% v/v crude oil plates. The purity of the strains as well as shape and size of the strains were analyzed using an Olympus CX21FS1 binocular microscope.

### Example 5: Gas Chromatography-mass Spectrometry (GC-MS) Analysis

Samples (1 ml) of enriched culture containing crude oil were extracted three times with ethyl acetate. Further, organic fraction was evaporated, dissolved in equal volume of chloroform and then analyzed by GC-MS analysis. GC coupled with a mass spectrometer from Agilent technologies (6890N series), injector unit (7683B series) was used. Further, MS system from Inert XL EI/CIMSD (5975B series) was also used. In addition, GC equipped with HP-5 column and helium as carrier gas, was also used. GC analysis started with an initial temperature of 55° C. held for 2 min, followed by an increase to 250° C. at a rate of 6° C./min, the temperature of 250° C. was maintained for 30 min resulting in a total run time of 65.5 min. An extract of about 1.0 microliters (μL) was injected using split-less mode. The MS analysis conditions were based on an inlet temperature of 250° C. and a mass to charge ratio (m/z) range of 15 m/z to 550 m/z.

### Example 6: Metagenomic DNA Extraction

Microbial DNA from the oil phase was recovered using two independent DNA extraction procedures. In the first procedure, briefly, 1 mL of the oil was vortex-mixed with 1 mL of sterile isooctane (2,2,4-trimethylpentane) and centrifuged at 5000×g for 15 min. Pellets were suspended in 1 mL of isooctane, vortexed and centrifuged again for 15 min. This washing step was repeated three times, and the resultant pellet was dried under vacuum. DNA was extracted from each dried pellet using a Zymo BIOMICS™ DNA Miniprep kit (Zymo Research). In the second procedure, an aliquot of 1 mL of crude oil was left overnight at 50° C. after mixing with an equal volume of sterile phosphate-buffered saline (PBS) solution having (8 grams (g) NaCl, 0.2 g KCl, 0.24 g KH₂PO₄ and 1.44 g Na₂HPO₄), per liter and further containing 0.1% polysorbate 20. The aqueous phase of the mixtures was recovered, filtered and microbial genomic DNA was extracted as previously described. DNA quantity and quality were measured using a Colibri Microvolume Spectrometer (Titertek Berthold, Pforzheim, Germany) in order to determine the concentration and purity of DNA (A260/280 and A260/230 ratios). DNA extracts obtained by these two methods were pooled to yield approximately 25 nanograms per microliter (ng/μl) of metagenomic DNA.

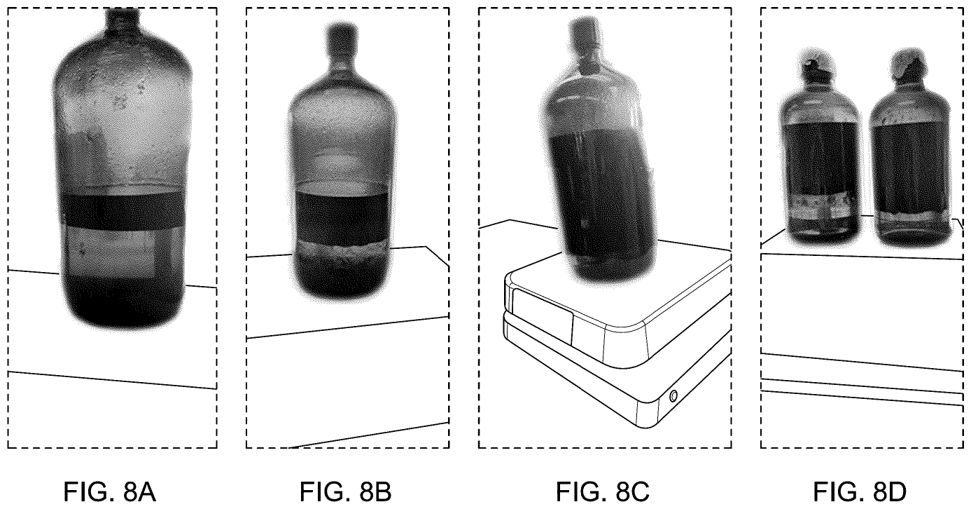

Isolation of halophilic crude oil bacterium was achieved. All enrichment cultures prepared in abovementioned examples were initiated in formation brine solution medium containing 25% v/v crude oil as the carbon and energy source. As can be seen from FIGS. 8A-8D, enrichment culture FB3, showed the presence of bacteria with diverse cellular morphologies and then was used for the isolation of pure bacterial strains in solid media. Three colonies were picked and a pure culture.

### Example 7: Materials and Methods with CO₂ (Culture Media Enrichment and Isolation Process)

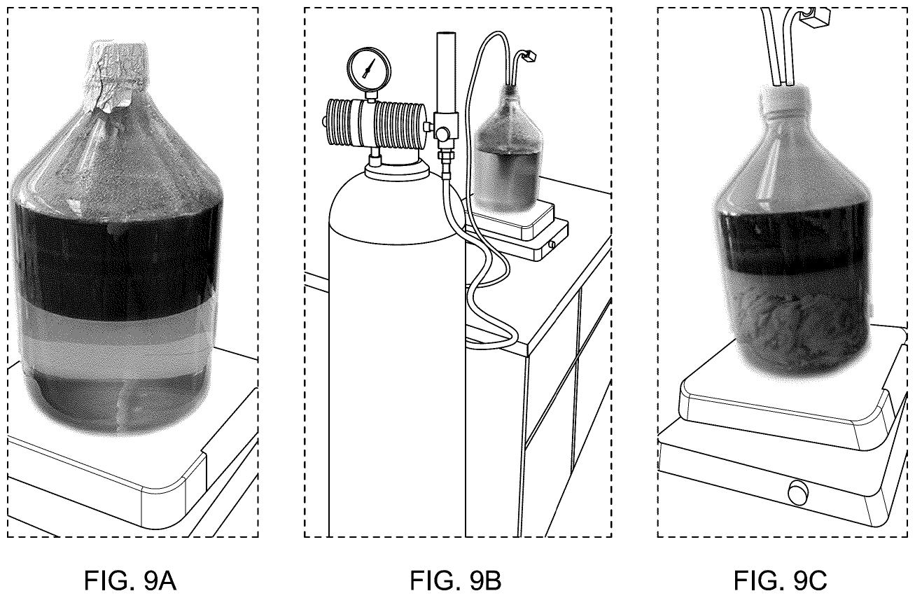

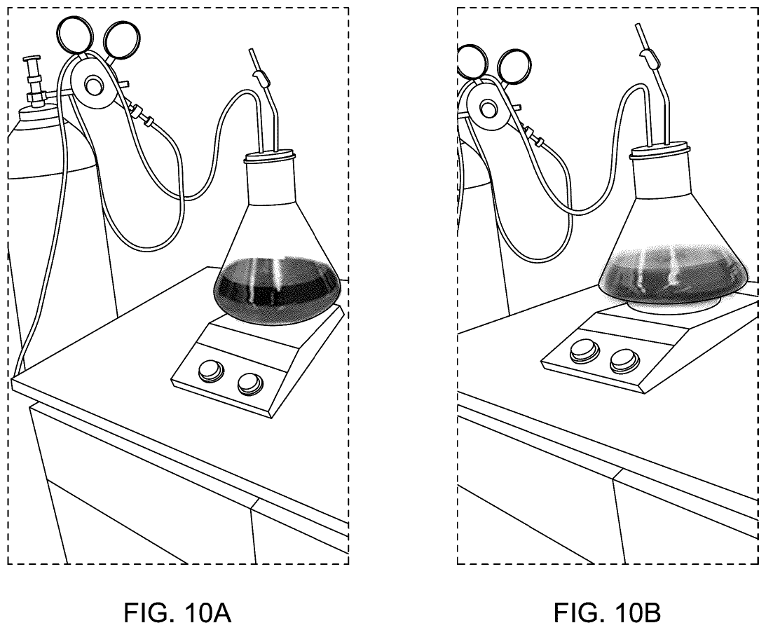

The Enrichment culture medium was prepared by using Saudi synthetic brine solution, NaCl concentration of 2.574603 mol/L. The formation brine solution was prepared through adding, 150.46 g NaCl, 0.5175g Na₂SO₄, 0.4874 g NaHCO₃, 69.823 g CaCl₂ dihydrate, 20.395 g MgCl₂(6H₂O) to 758.32 ml distilled water and 25% v/v commercial plant fatty oil products (total fat content of about 100 g with saturated fat at 15.35 g, monounsaturated fat at 20.45 g, polyunsaturated fat at 30.55 g, and trans-fat at 0.5 g per 100 ml). The medium was sterilized by autoclaving at 121° C. for 20 min. Further, at room temperature, the crude oil was added at 25% v/v. The medium was assessed by using borosilicate glass flat bottom flask having a volume of 5000 ml with a working volume of 4000 mL, as shown in FIG. 9A. Furthermore, the contents of the flask were continuously stirred at 200 rpm by using a magnetic stirrer at room temperature of about 20° C. and deoxygenated by bubbling a CO₂ gas, as shown in FIG. 9B. The anaerobic condition of the culture medium was maintained, and the flasks were sealed with rubber stoppers with adhesive tape in continuous mode for at least 90 days, as shown in FIG. 9C. Moreover, 10 ml of samples (every ten days) were collected for analysis, which included isolating the individual strains, metagenomics, and GC-MS analyses. Outstanding samples were stored at −20° C. until further tests were conducted.

For enrichment, 1 ml sample was isolated on solid medium. Aliquots with a dilution of 0.1 ml were plated onto agar formation brine medium with crude oil 1% v/v as carbon source. The plates were incubated at 30° C. for 3 days, until colony formation started. Singular colonies were picked and used for screening. Individual colonies were purified by repeated streaking on agar formation brine medium containing 1% v/v crude oil plates. The purity of the strains as well as shape and size of the strains were analyzed using an Olympus CX21FS1 binocular microscope.

### Example 8: GC-MS Analysis

Samples (1 ml) of enriched culture containing crude oil were extracted three times with ethyl acetate. Further, organic fraction was evaporated, dissolved in equal volume of chloroform and then analyzed by GC-MS analysis. GC coupled with a mass spectrometer from Agilent technologies (6890N series), injector unit (7683B series) was used. Further, MS system from Inert XL EI/CIMSD (5975B series) was also used. In addition, GC equipped with HP-5 column and helium as carrier gas, was also used. GC analysis started with an initial temperature of 55° C. held for 2 min, followed by an increase to 250° C. at a rate of 6° C./min, the temperature of 250° C. was maintained for 30 min resulting in a total run time of 65.5 min. An extract of about 1.0 μL was injected using split-less mode. The MS analysis conditions were based on an inlet temperature of 250° C. and a mass to charge ratio range of 15 m/z to 550 m/z.

### Example 9: Metagenomic DNA Extraction

Microbial DNA from the oil phase was recovered using two independent DNA extraction procedures. In the first procedure, briefly, 1 mL of the oil was vortex-mixed with 1 mL of sterile isooctane (2,2,4-trimethylpentane) and centrifuged at 5000×g for 15 min. Pellets were suspended in 1 mL of isooctane, vortexed and centrifuged again for 15 min. This washing step was repeated three times, and the resultant pellet was dried under vacuum. DNA was extracted from each dried pellet using a Zymo BIOMICS™ DNA Miniprep kit (Zymo Research). In the second procedure, an aliquot of 1 mL of crude oil was left overnight at 50° C. after mixing with an equal volume of sterile phosphate-buffered saline (PBS) solution having (8 g NaCl, 0.2 g KCl, 0.24 g KH₂PO₄ and 1.44 g Na₂HPO₄), per liter and further containing 0.1% polysorbate 20. The aqueous phase of the mixtures was recovered, filtered and microbial genomic DNA was extracted as previously described. DNA quantity and quality were measured using a Colibri Microvolume Spectrometer (Titertek Berthold, Pforzheim, Germany) in order to determine the concentration and purity of DNA (A260/280 and A260/230 ratios). DNA extracts obtained by these two methods were pooled to yield approximately 25 ng/μl of metagenomic DNA.

### Example 10: Materials and Methods with Nitrogen Gas (N₂) (Culture Media and Isolation Procedure)

The Enrichment culture medium was prepared by using Saudi synthetic brine solution, NaCl concentration of 2.574603 mol/L. The formation brine solution was prepared through adding, 150.46 g NaCl, 0.5175 g Na₂SO₄, 0.4874 g NaHCO₃, 69.823 g CaCl₂ dihydrate, 20.395 g MgCl₂(6H₂O) to 758.32 ml distilled water and 25% v/v commercial plant fatty oil products (total fat content of about 100 g with saturated fat at 15.35 g, monounsaturated fat at 20.45 g, polyunsaturated fat at 30.55 g, and trans-fat at 0.5g per 100 ml). The medium was sterilized by autoclaving at 121° C. for 20 min. Further, at room temperature, the crude oil was added at 25% v/v. The medium was assessed by using borosilicate flat bottom, Erlenmeyer flask (2500 ml) with a working volume of 2000 mL, as shown in FIG. 10A. Further, the contents of the flask were continuously stirred at 200 rpm by using a magnetic stirrer at room temperature of about 25° C. and deoxygenated by bubbling N₂ gas (99%), as shown in FIG. 10B. The anaerobic condition of the culture medium was maintained, and the flasks were sealed with rubber stoppers with adhesive tape in continuous mode for more than 90 days. 10 ml of samples (every ten days) were collected for analysis, which included isolating the individual strains, metagenomics and GC-MS analyses. Outstanding samples were stored at −20° C. until further tests were conducted.

For enrichment, 1 ml sample was isolated on solid medium. Aliquots with a dilution of 0.1 ml were plated onto agar formation brine medium with crude oil 1% v/v as carbon source. The plates were incubated at 30° C. for 3 days, until colony formation started. Singular colonies were picked and used for screening. Individual colonies were purified by repeated streaking on agar formation brine medium containing 1% v/v crude oil plates. The purity of the strains as well as shape and size of the strains were analyzed using an Olympus CX21FS1 binocular microscope.

### Example 11: GC-MS Analysis

Samples (1 ml) of enriched culture containing crude oil were extracted three times with ethyl acetate. Further, organic fraction was evaporated, dissolved in equal volume of chloroform and then analyzed by GC-MS analysis. GC coupled with a mass spectrometer from Agilent technologies (6890N series), injector unit (7683B series) was used. Further, MS system from Inert XL EI/CIMSD (5975B series) was also used. In addition, GC equipped with HP-5 column and helium as carrier gas, was also used. GC analysis started with an initial temperature of 55° C. held for 2 min, followed by an increase to 250° C. at a rate of 6° C./min, the temperature of 250° C. was maintained for 30 min resulting in a total run time of 65.5 min. An extract of about 1.0 μL was injected using split-less mode. The MS analysis conditions were based on an inlet temperature of 250° C. and a mass to charge ratio range of 15 m/z to 550 m/z.

### Example 12: Metagenomic DNA Extraction

Microbial DNA from the oil phase was recovered using two independent DNA extraction procedures. In the first procedure, briefly, 1 mL of the oil was vortex-mixed with 1 mL of sterile isooctane (2,2,4-trimethylpentane) and centrifuged at 5000×g for 15 min. Pellets were suspended in 1 mL of isooctane, vortexed and centrifuged again for 15 min. This washing step was repeated three times, and the resultant pellet was dried under vacuum. DNA was extracted from each dried pellet using a Zymo BIOMICS™ DNA Miniprep kit (Zymo Research). In the second procedure, an aliquot of 1 mL of crude oil was left overnight at 50° C. after mixing with an equal volume of sterile phosphate-buffered saline (PBS) solution having (8 g NaCl, 0.2 g KCl, 0.24 g KH₂PO₄ and 1.44 g Na₂HPO₄), per liter and further containing 0.1 % polysorbate 20. The aqueous phase of the mixtures was recovered, filtered and microbial genomic DNA was extracted as previously described. DNA quantity and quality were measured using a Colibri Microvolume Spectrometer (Titertek Berthold, Pforzheim, Germany) in order to determine the concentration and purity of DNA (A260/280 and A260/230 ratios). DNA extracts obtained by these two methods were pooled to yield approximately 25 ng/μl of metagenomic DNA.

Isolation of halophilic crude oil bacterium, all enrichment cultures initiated in formation brine solution medium containing 25% (v/v) crude oil as the carbon and energy source, became turbid and the dark layer of crude oil became complete homogeneous, indicating the microbial growth of this substrate. Enrichment culture N₂, showed the presence of bacteria with diverse cellular morphologies and then was used for the isolation of pure bacterial strains in solid media.

The aspects of the present disclosure provide the artificial lung system 100, 300. In particular, the artificial lung system 100 is designed to support patients with respiratory failure and pulmonary illnesses. The artificial lung system 100 may be configured for direct support in the parallel configuration 400 and supplemental support in the series configuration 450. Further, the artificial lung system 100 is constructed to cause least amount of damage to blood cells during employment. Injury to cells may result in clotting and an immune response which is to be avoided when supporting a respiratory failure patient. Hence, a shear stress of the system 100 should remain within predetermined bounds. Furthermore, in some implementations, a large scale artificial lung system 100 may also be used for direct air carbon capture from the atmosphere in order to reduce CO₂ levels. The artificial lung system 100 as described herein, with additional key components may be used to provide solutions in order to address challenges in gas exchange, wound healing via the biocompatible properties of spider silk, and the metal switching capabilities of cadmium containing carbonic anhydrase. Furthermore, the culture media enrichment as explained in the examples section may provide favorable results in the field of oil recovery and oil bioremediation. As described with respect to the examples provided in the present disclosure, the microorganisms may be useful to perform Calvin-Benson pathway. The pathway encompasses a series of enzymatic reactions resulting in the conversion of carbon dioxide into glucose. To summarize, the present disclosure may provide effective and efficient solutions for biomedical support devices, in addition, in some cases it may provide direct air carbon capture functionality as well. The carbon capture by the aforementioned microorganisms may be applied to microbial enhanced oil recovery, by utilizing lipid-based nutrients and brine water to enhance the oil recovery. As such, the microorganisms may assist in mobilizing trapped oil particles and an introduction of CO₂ or N₂ in a particular oil reservoir, may be used to further assist the oil recovery.

### Example 13: Carbon Fixation Pathways

The microorganisms can be engineered to enhance the natural process of carbon fixation, which may mitigate the impacts of climate change by converting CO₂ from the atmosphere into organic compounds. Three primary cycles, the CETCH cycle, THETA cycle, and Calvin-Benson cycle, each represent different biochemical pathways for carbon fixation:
1. CETCH Cycle (Crotonyl-CoA/ethylmalonyl-CoA/hydroxybutyryl-CoA Cycle): It operates in some bacteria and is used to fix carbon more efficiently under certain conditions. Genetic engineering can enhance this cycle to increase its efficiency or adapt it to different organisms or environmental conditions.
2. THETA Cycle (Tetrahydrofolate cycle): This cycle involves the fixation of CO₂ through the metabolism of tetrahydrofolate. Its different enzyme mechanisms and reaction steps could be exploited to improve CO₂ fixation in various industrial applications.
3. Calvin-Benson Cycle: It occurs in the chloroplasts of photosynthetic organisms like plants, algae, and cyanobacteria. Genetic engineering efforts here often focus on enhancing the activity and efficiency of key enzymes such as RuBisCO, or introducing the cycle into non-photosynthetic organisms to create autotrophic microbes that can fix carbon.

For genetically engineered microorganisms, these pathways in microorganisms dependent on waste, the focus would be on integrating these carbon fixation pathways into the metabolic processes of organisms that can also utilize waste products as a source of energy and nutrients. This could involve using waste from agricultural, industrial, oil, or municipal sources to feed these engineered microbes, thereby achieving dual benefits of waste reduction and CO₂ fixation.

Such engineered microbes could be used in various applications, including biofuel production, biodegradation, and as part of carbon capture and storage (CCS) technologies. The engineering process might involve techniques such as CRISPR-Cas9 for precise edits to the microbial genome, selection of promoter regions to control gene expression, and metabolic modeling for enhancing pathway efficiencies.

Numerous modifications and variations of the present disclosure are possible in light of the above teachings. It is therefore to be understood that within the scope of the appended claims, the invention may be practiced otherwise than as specifically described herein.

1. An artificial lung system for direct air carbon capture, comprising:
a microfluidic device having a first connection point, a second connection point, and a third connection point, wherein the microfluidic device is configured to perform the direct air carbon capture;
a plurality of air sacs including a posterior sac and an anterior sac each having an inlet and an outlet;
a plurality of air channels each including a valve, wherein the valve of each air channel of the plurality of air channels is configured to regulate microfluidic flows in the artificial lung system; and
an artificial heart fluidly connected to the third connection point of the microfluidic device and configured to exchange a fluid in the microfluidic device,
wherein the inlet of the posterior sac is connected to a first air channel of the plurality of air channels via a first valve and the outlet of the posterior sac is connected to the first connection point of the microfluidic device; and wherein the inlet of the anterior sac is connected to the second connection point of the microfluidic device and the outlet of the anterior sac is connected to a second air channel of the plurality of air channels via a second valve.

2. The artificial lung system of claim 1, wherein each air sac of the plurality of air sacs further comprises a plurality of biomimetic soft tissues and a plurality of biomimetic hard tissues.

3. The artificial lung system of claim 2, wherein each biomimetic soft tissue of the plurality of biomimetic soft tissues comprises a spider silk.

4. The artificial lung system of claim 3, wherein each biomimetic hard tissue of the plurality of biomimetic hard tissues comprises at least one carbonic anhydrase immobilized in a solid matrix.

5. The artificial lung system of claim 4, wherein the carbonic anhydrase is a cadmium-containing carbonic anhydrase (CDCA).

6. The artificial lung system of claim 1, wherein the first valve and the second valve are air solenoid valves.

7. The artificial lung system of claim 6, wherein the artificial lung system is configured such that the first valve is open, and the second valve is closed during an inhale phase.

8. The artificial lung system of claim 6, wherein the artificial lung system is configured such that the first valve is closed, and the second valve is open during an exhale phase.

9. The artificial lung system of claim 2, wherein the posterior sac and the anterior sac form an origamilike bellow.

10. The artificial lung system of claim 9, wherein the origamilike bellow comprises the plurality of biomimetic soft and hard tissues.

11. The artificial lung system of claim 10, wherein the fluid contains one or more genetically engineered microorganisms.

12. The artificial lung system of claim 11, wherein the one or more genetically engineered microorganisms have a carbon fixation pathway selected from the group consisting of CETCH cycle, THETA cycle, and Calvin-Benson cycle.

13. The artificial lung system of claim 1, further comprising a peristaltic pump connected to the plurality of air channels, wherein the peristaltic pump is configured to control an air flow of the artificial lung system.

14. The artificial lung system of claim 1, wherein the microfluidic device is a flexible microfluidic device having a shape of alveoli.

15. The artificial lung system of claim 1, wherein the microfluidic device comprises:
a plurality of distribution channels each having a first end and a second end;
a first distributor connected to the first end of each distribution channel of the plurality of distribution channels, wherein the first distributor includes a plurality of first connectors connected to the first connection point of the microfluidic device; and
a second distributor connected to the second end of each distribution channel of the plurality of distribution channels, wherein the second distributor includes a plurality of second connectors connected to the second connection point of the microfluidic device,
wherein the plurality of distribution channels is configured for gas exchange.

16. The artificial lung system of claim 15, wherein each distribution channel of the plurality of distribution channels is semi-elliptical and wherein the plurality of distribution channels is arranged in an approximately concentric circular shape.

17. The artificial lung system of claim 1, wherein the one or more air sacs is configured to support a unidirectional airflow.
